(19) United States
(12) Patent Application Publication
(10) Pub. No.: US 2023/0063356 A1
(43) Pub. Date: Mar. 2, 2023

(54) BIOLOGICAL SUBSTANCE DETECTION CHIP, BIOLOGICAL SUBSTANCE DETECTION DEVICE AND BIOLOGICAL SUBSTANCE DETECTION SYSTEM

(71) Applicant: SONY SEMICONDUCTOR SOLUTIONS CORPORATION, KANAGAWA (JP)

(72) Inventors: HARUMI TANAKA, KANAGAWA (JP); YOSHIAKI MASUDA, KANAGAWA (JP); YUSUKE UESAKA, KANAGAWA (JP); TAKAFUMI MORIKAWA, KANAGAWA (JP)

(21) Appl. No.: 17/904,161

(22) PCT Filed: Jan. 29, 2021
(86) PCT No.: PCT/JP2021/003239

§ 371 Date: Aug. 12, 2022

(30) Foreign Application Priority Data
Feb. 19, 2020 (JP) 2020-026052

(51) Int. Cl. B01L 3/00; G01N 21/64; H01L 27/146

(52) U.S. Cl. CPC B01L 3/502715; G01N 21/64; H01L 27/14629; B01L 2300/0636

(57) ABSTRACT

There is provided a biological substance detection chip having high detection accuracy. The present technology provides a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit. The present technology also provides a biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit.

### TECHNICAL FIELD

The present technology relates to a biological substance detection chip, a biological substance detection device and a biological substance detection system.

### BACKGROUND ART

In recent years, technical research on gene analysis, protein analysis, cell analysis and the like has progressed in various fields such as medicine, drug discovery, clinical examination, food, agriculture, and engineering. In particular, recently, the development and practical application of detection technology on chips such as lab-on-a-chip in which various reactions such as detection and analysis of biological substances such as nucleic acids, proteins, cells, and microorganisms are performed in microscale channels and wells provided in the chips have been progressed. These are being focused on as a method of easily measuring biological substances and the like.

For example, PTL 1 discloses an optical detection device including at least a first substrate in which a plurality of wells are formed, a second substrate in which a heating unit is provided so that it comes in contact with the wells, a third substrate in which a plurality of light emitting units are positioned to correspond to the positions of the wells, and a fourth substrate in which a plurality of light detecting units are positioned to correspond to the positions of the wells. In this optical detection device, various reactions that proceed in the wells can be measured.

In addition, for example, PTL 2 discloses a chemical sensor including a substrate in which an optical detection unit is formed, and a plasmon absorption layer laminated on the substrate and having a metal nanostructure that causes plasmon absorption. This chemical sensor can detect emission of light caused by binding between a probe material fixed on the sensor and a target material.

### CITATION LIST

### Patent Literature

[PTL 1]

JP 2010-284152A

[PTL 2]

WO 2013/080473

### SUMMARY

### Technical Problem

In a plurality of regions (for example, a plurality of wells) on a chip, when light emitted from a biological substance is detected using a plurality of light detection units corresponding to the regions, problems such as light leakage from regions adjacent to the optical detection units occur. For example, in the regions, when different reactions are caused to proceed and emission of light caused by respective reactions is detected, erroneous determination may be made when emission of light from other regions is erroneously detected.

In addition, in a general image sensor for imaging, an imaging object is set apart from a sensor, the angle of incidence of light from the imaging object to the surface of the sensor is about 0 to 30 degrees, and light can be condensed on a photoelectric conversion unit such as an on-chip lens on the surface of the sensor. However, when biological substances such as DNA, proteins such as antibodies, cells and the like held on the surface of the chip are detected, since light is emitted in all directions from the biological substances, the amount of light that can be acquired by the photoelectric conversion unit is about 10 to 30% of the total, and in a light condensing structure of a general image sensor for imaging and a DNA sensor from which an on-chip lens is removed, there are problems that emission of light from biological substances cannot be effectively used, and the detection accuracy is lowered.

Therefore, a main object of the present technology is to provide a biological substance detection chip having high detection accuracy.

### Solution to Problem

Specifically, first, the present technology provides a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit. The present technology also provides a biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit.

In the biological substance detection chip according to the present technology, a wiring layer may be provided below the photoelectric conversion unit.

In addition, a reflective layer may be provided below the photoelectric conversion unit.

In the biological substance detection chip according to the present technology, the light guiding unit may be composed of a refractive member and/or a reflective member provided between the pixels.

In addition, a recess formed on the holding surface may be used as the light guiding unit.

In the biological substance detection chip according to the present technology, signal charges from the plurality of pixels may be added and output. Regarding the biological substance that can be detected by the biological substance detection chip according to the present technology, one or more biological substances selected from among nucleic acids, proteins, cells, microorganisms, chromosomes, ribosomes, mitochondria, organelles (cell organelles), and complexes thereof may be exemplified.

Next, the present technology provides a biological substance detection device including: a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit; and an analysis unit that analyzes electrical information acquired by the biological substance detection chip.

In addition, the present technology provides a biological substance detection device including a biological substance detection chip which is composed of a plurality of pixels, in which the pixel includes a holding surface on which a biological substance is held and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit; and an analysis unit that analyzes electrical information acquired by the biological substance detection chip.

The present technology also provides a biological substance detection system including a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit; and an analysis device that analyzes electrical information acquired by the biological substance detection chip.

In addition, the present technology provides a biological substance detection system including a biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit, and an analysis device that analyzes electrical information acquired by the biological substance detection chip.

In the present technology, “biological substance” widely includes nucleic acids, proteins, cells, microorganisms, chromosomes, ribosomes, mitochondria, organelles (cell organelles), complexes thereof, and the like. Cells include animal cells (such as blood cell lineage cells) and plant cells. Microorganisms include bacteria such as E. coli, viruses such as tobacco mosaic virus, and fungi such as yeast.

### BRIEF DESCRIPTION OF DRAWINGS

FIG. 1 is a schematic conceptual view schematically showing interactions between biological substances S that can be detected by a biological substance detection chip 1, a biological substance detection device 2, and a biological substance detection system 3 according to the present technology.

FIG. 2 is a schematic conceptual view schematically showing interactions between biological substances S that can be detected by the biological substance detection chip 1, the biological substance detection device 2, and the biological substance detection system 3 according to the present technology.

FIG. 3 is a schematic conceptual view schematically showing interactions between biological substances S that can be detected by the biological substance detection chip 1, the biological substance detection device 2, and the biological substance detection system 3 according to the present technology.

FIG. 4 is a schematic conceptual view schematically showing screening of other substances that can be performed by the biological substance detection chip 1, the biological substance detection device 2, and the biological substance detection system 3 according to the present technology.

FIG. 5 is a schematic conceptual view schematically showing screening of other substances that can be performed by the biological substance detection chip 1, the biological substance detection device 2, and the biological substance detection system 3 according to the present technology.

FIG. 6 is a schematic conceptual view schematically showing screening of other substances that can be performed by the biological substance detection chip 1, the biological substance detection device 2, and the biological substance detection system 3 according to the present technology.

FIG. 7 is a schematic end view schematically showing a first embodiment of the biological substance detection chip 1 according to the present technology.

FIG. 8 is a schematic end view schematically showing an example of a photoelectric conversion unit 112 and a wiring layer 113 of a pixel 11 in the first embodiment of the biological substance detection chip 1 according to the present technology.

FIG. 9 is a schematic bottom view of an example of the photoelectric conversion unit 112 and the wiring layer 113 when viewed from the side of the wiring layer 113.

FIG. 10 is a schematic end view schematically showing a second embodiment of the biological substance detection chip 1 according to the present technology.

FIG. 11 is a schematic end view schematically showing an example of the photoelectric conversion unit 112 and the wiring layer 113 of the pixel 11 in the second embodiment of the biological substance detection chip 1 according to the present technology.

FIG. 12 is a schematic end view schematically showing a third embodiment of the biological substance detection chip 1 according to the present technology.

FIG. 13 is a schematic end view schematically showing a fourth embodiment of the biological substance detection chip 1 according to the present technology.

FIG. 14 is a schematic end view schematically showing a first modified example of the fourth embodiment of the biological substance detection chip 1 according to the present technology.

FIG. 15 is a schematic end view schematically showing a second modified example of the fourth embodiment of the biological substance detection chip 1 according to the present technology.

FIG. 16 is a schematic perspective view schematically showing a planar layout of the fourth embodiment of the biological substance detection chip 1 according to the present technology.

FIG. 17 is a schematic perspective view schematically showing a first modified example of the planar layout of the fourth embodiment of the biological substance detection chip 1 according to the present technology.

FIG. 18 is a schematic perspective view schematically showing a second modified example of the planar layout of the fourth embodiment of the biological substance detection chip 1 according to the present technology.

FIG. 19 is a schematic end view schematically showing a fifth embodiment of the biological substance detection chip 1 according to the present technology.

FIG. 20 is a schematic end view schematically showing a first modified example of the fifth embodiment of the biological substance detection chip 1 according to the present technology.

FIG. 21 is a schematic end view schematically showing a sixth embodiment of the biological substance detection chip 1 according to the present technology.

FIG. 22 is a schematic end view schematically showing a first modified example of the sixth embodiment of the biological substance detection chip 1 according to the present technology.

FIG. 23 is a schematic end view schematically showing a second modified example of the sixth embodiment of the biological substance detection chip 1 according to the present technology.

FIG. 24 is a schematic end view schematically showing a seventh embodiment of the biological substance detection chip 1 according to the present technology.

FIG. 25 is a schematic end view schematically showing a first modified example of the seventh embodiment of the biological substance detection chip 1 according to the present technology.

FIG. 26 is a schematic end view schematically showing a first modified example of the seventh embodiment of the biological substance detection chip 1 according to the present technology.

FIG. 27 is a schematic bottom view schematically showing the biological substance detection chip 1, which is an example of the embodiments so far, when viewed from the side of the wiring layer 113.

FIG. 28 is a schematic end view taken along the line A-A, schematically showing the biological substance detection chip 1, which is an example of the embodiments so far.

FIG. 29 is a schematic end view taken along the line B-B, schematically showing the biological substance detection chip 1, which is an example of the embodiments so far.

FIG. 30 is an equivalent circuit diagram showing an example of the configuration of FIG. 27.

FIG. 31 is a schematic end view taken along the line A-A, schematically showing a modified example of FIG. 28.

FIG. 32 is an equivalent circuit diagram of an eighth embodiment of the biological substance detection chip 1 according to the present technology.

FIG. 33 is a block diagram showing a concept of the biological substance detection device 2 according to the present technology.

FIG. 34 is a block diagram showing a concept of the biological substance detection system 3 according to the present technology.

### DESCRIPTION OF EMBODIMENTS

Hereinafter, preferable embodiments for implementing the present technology will be described with reference to the drawings. The embodiments described below show examples of representative embodiments of the present technology, but the scope of the present technology should not be narrowly understood based on the embodiments. Here, description will proceed in the following order.

1. Overview of Biological Substance Detection Performed by Present Technology

(1) Detection of Biological Substance S Itself

(2) Detection of Interactions of Biological Substance S

(3) Screening of Other Substances

2. Biological Substance Detection Chip 1

(1) First Embodiment

(2) Second Embodiment

(3) Third Embodiment

(4) Fourth Embodiment

(5) Fifth Embodiment

(6) Sixth Embodiment

(7) Seventh Embodiment

(8) Eighth Embodiment

3. Biological Substance Detection Device 2

4. Biological Substance Detection System 3

<1. Overview of Biological Substance Detection Performed by Present Technology>An overview of detection of a biological substance S performed by a biological substance detection chip 1, a biological substance detection device 2, and a biological substance detection system 3 according to the present technology will be described. The biological substance detection chip 1 and the biological substance detection device 2, and the biological substance detection system 3 according to the present technology can be used for (1) detection of a biological substance S itself, (2) detection of interactions of the biological substance S, (3) screening of other substances (for example, medicinal components) using biological substance S and the like. Here, each detection is performed on a holding surface 111 of the biological substance detection chip 1 to be described below.

(1) Detection of Biological Substance S Itself

For example, the present technology can be used for detecting bio-substances such as red blood cells, white blood cells, platelets, cytokines, hormone substances, sugars, lipids, proteins and the like contained in body fluids such as blood, urine, feces, and saliva; microorganisms such as bacteria, fungi, viruses and the like contained in body fluids and water; and genes in cells and microorganisms. For example, after staining with a dye that acts specifically on a detection target substance or a non-detection target substance, the presence of a detection target substance can be detected depending on the presence of desired light detection. The detection results can be used for disease diagnosis, internal environment diagnosis, water quality examination and the like.

(2) Detection of Interactions of Biological Substance S

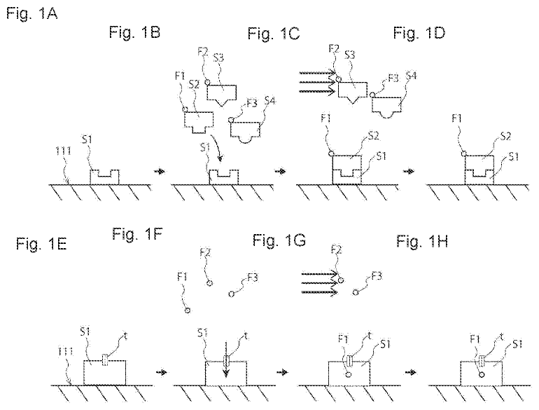

For example, the present technology can be used to detect interactions such as protein interactions, nucleic acid hybridization, and binding of cytokines and hormone substances to receptors. Specific detection examples will be described with reference to FIGS. 1 to 3.

For example, as shown in A to D in FIG. 1, a biological substance 51 such as a protein or a receptor (or an imitation of a receptor) is fixed on the holding surface 111 (refer to A in FIG. 1), and fixed dyes such as fluorescent dyes F1 to F3 are added to biological substances S2 to S4 for checking the interaction thereof (refer to B in FIG. 1). Then, the biological substances S3 and S4 that do not interact with the biological substance 51 are washed off (refer to C in FIG. 1), and the interaction between the biological substance 51 and the biological substance S2 can be detected by detecting the fluorescent dye F1 from the holding surface 111 (refer to D in FIG. 1).

For example, as shown in E to H in FIG. 1, the biological substance 51 such as a cell is fixed on the holding surface 111, and an entrapped illuminant F1 can be detected via a transporter t (for example, a transporter in a cell membrane) of the biological substance 51.

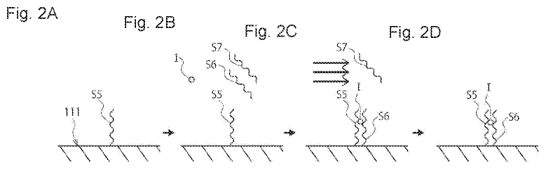

For example, as shown in A to D in FIG. 2, a probe S5 composed of DNA, RNA or the like is fixed to the holding surface 111 (refer to A in FIG. 2), and a sample containing DNA S6 and S7 that can be targets, and an intercalator I are added (refer to B in FIG. 2). Then, when the DNA S6 having a sequence complementary to the probe S5 is contained in the sample, a hybridization reaction occurs. The DNA S7 that is not hybridized is washed off (refer to C in FIG. 2), and hybridization between the probe S5 and the target DNA S6 can be detected by detecting light from the intercalator I from the holding surface 111 (refer to D in FIG. 2).

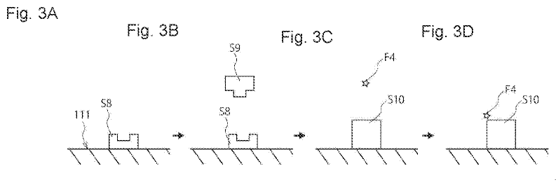

For example, as shown in A to D in FIG. 3, a biological substance S8 is fixed on the holding surface 111(refer to A in FIG. 3), and a biological substance S9 that interacts with the biological substance S8 to form a new substance S10 is added (refer to B in FIG. 3). Next, a dye such as a fluorescent dye F4 that specifically binds to the substance S10 is added (refer to C in FIG. 3), and the fluorescent dye F4 is detected from the holding surface 111 (refer to D in FIG. 3), and thus the interaction between the biological substance S8 and the biological substance S9 can be detected.

(3) Screening of Other Substances

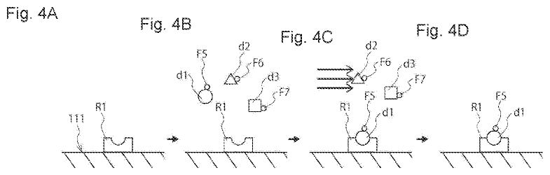

For example, the present technology can be used for screening of substances that can be agonists or antagonists of various receptors, and screening of agents for inhibiting production of various microorganisms, antibacterial agents, bactericidal agents and the like. Specific detection examples will be described with reference to FIGS. 4 to 6.

For example, as shown in A to D in FIG. 4, a receptor R1 (or an imitation of the receptor R1) is fixed on the holding surface 111 (refer to A in FIG. 4), and fixed dyes such as fluorescent dyes F5 to F7 are added to substances d1 to d3 for checking operability of the receptor R1 (refer to B in FIG. 4). Then, the substances d2 and d3 that do not bind to the receptor R1 are washed off (refer to C in FIG. 4), and it is possible to perform screening of the substance d1 that can be an agonist of the receptor R1 by detecting the fluorescent dye F5 from the holding surface 111 (refer to D in FIG. 3).

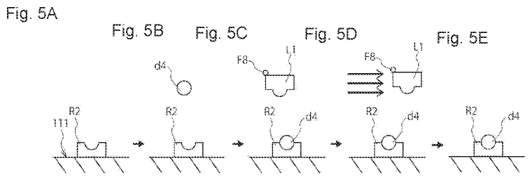

For example, as shown in A to E in FIG. 5, a receptor R2 (or an imitation of the receptor R2) is fixed on the holding surface 111 (refer to A in FIG. 5), and a substance d4 for checking antagonism of the receptor R2 is added (refer to B in FIG. 5). Next, a ligand L1 that binds to the receptor R2 to which a dye such as a fluorescent dye F8 is fixed is added (refer to C in FIG. 5). In this case, if the substance d4 can be an antagonist of the receptor R2, the ligand L1 cannot bind to the receptor R2 because the receptor R2 and the substance d4 are already bound to each other (refer to C in FIG. 5). In this state, after the ligand L1 that does not bind to the receptor R2 is washed off (refer to D in FIG. 5), even if an attempt is made to detect the fluorescent dye F8 from the holding surface 111, light is not detected because the fluorescent dye F8 is not present on the holding surface 111 due to the washing off (refer to E in FIG. 5).

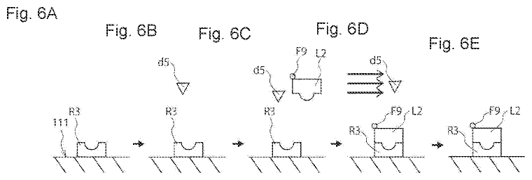

On the other hand, for example, as shown in A to E in FIG. 6, a receptor R3 (or an imitation of the receptor R3) is fixed on the holding surface 111 (refer to A in FIG. 6), and a substance d5 for checking antagonism of the receptor R3 is added (refer to B in FIG. 6). Next, a ligand L2 that binds to the receptor R3 to which a dye such as a fluorescent dye F9 is fixed is added (refer to C in FIG. 6). In this case, when the substance d5 cannot be an antagonist of the receptor R3, the ligand L2 binds to the receptor R3 (refer to D in FIG. 6). In this state, when the substance d5 that does not bind to the receptor R3 is washed off (refer to D in FIG. 6), the fluorescent dye F9 is detected from the holding surface 111 (refer to E in FIG. 6).

In this manner, as shown in FIGS. 5 and 6, it is possible to perform screening of the substance d4 that can be an antagonist of the receptor R3 depending on whether the fluorescent dye F8 or the fluorescent dye F9 is detected from the holding surface 111.

<2. Biological Substance Detection Chip 1>

### (1) First Embodiment

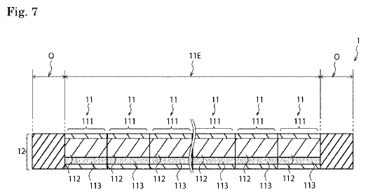

FIG. 7 is a schematic end view schematically showing a first embodiment of the biological substance detection chip 1 according to the present technology. The biological substance detection chip 1 according to the first embodiment has an effective pixel region 11E in which a plurality of pixels 11 are two-dimensionally arranged in a matrix. Each pixel 11 includes at least a holding surface 111 on which a biological substance S is held, a photoelectric conversion unit 112 provided on a semiconductor substrate 12, and a wiring layer 113.

The holding surface 111 is not particularly limited as long as it has a configuration that can hold the biological substance S, and a surface treatment can be freely used. For example, the holding surface 111 can be formed by applying a photosensitive silane coupling agent or the like that is modified with ultraviolet ray emission to be hydrophilic and selectively emitting ultraviolet rays to a region in which the biological substance S is desired to be held. In addition, for example, when the holding surface 111 is treated with avidin, the biological substance S such as a nucleic acid whose one end is biotinylated can be held by an avidin-biotin bond. In addition, according to the configuration in which a liquid can be held on the holding surface 111, it is also possible to hold the biological substance S in the liquid.

In the photoelectric conversion unit 112, for example, a photoelectric conversion element such as a photodiode can be freely used. A circuit used in a general image sensor can be provided in the wiring layer 113.

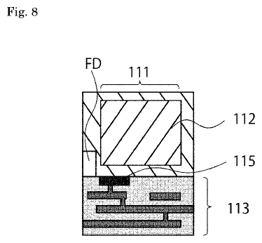

FIG. 8 shows an example of the photoelectric conversion unit 112 and the wiring layer 113 of the pixel 11. There are provided a transfer transistor gate 115 that transfers charges of the photoelectric conversion unit, and an amplifier transistor gate 116, a selection transistor gate 117, and a reset transistor gate 118 (which are not shown), which are connected by multilayer wiring in the wiring layer 113.

In addition, although not shown, an optical black pixel, a wiring region, and the like can be provided on the outside O of the effective pixel region 11E.

In the first embodiment, in this order from above the biological substance detection chip 1, the holding surface 111→the photoelectric conversion unit 112→the wiring layer 113 are arranged in that order. In this manner, when the photoelectric conversion unit 112 is provided above the wiring layer 113, since the distance between the holding surface 111 and the photoelectric conversion unit 112 is short compared to the chip in which the holding surface 111→the wiring layer 113→the photoelectric conversion unit 112 are arranged in that order, the photoelectric conversion unit 112 can utilize a larger amount of light emitted from the biological substance S. As a result, it is possible to improve detection accuracy.

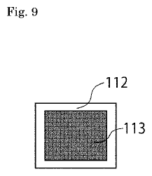

In addition, as shown in FIG. 9, when a part of the multilayer wiring of the wiring layer 113 is formed into a solid pattern, light emitted from the biological substance S can be reflected and returned to the photoelectric conversion unit 112, and the photoelectric conversion unit 112 can utilize a larger amount of light. As a result, it is possible to further improve detection accuracy.

### (2) Second Embodiment

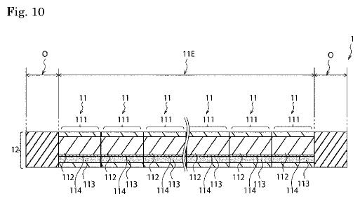

FIG. 10 is a schematic end view schematically showing a second embodiment of the biological substance detection chip 1 according to the present technology. In the second embodiment, a reflective layer 114 is provided below the photoelectric conversion unit 112.

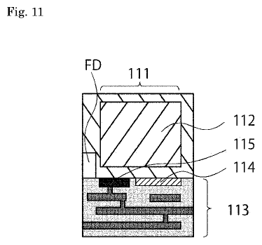

FIG. 11 shows an example of the photoelectric conversion unit 112, the reflective layer 114, and the wiring layer 113 of the pixel 11. There are provided the transfer transistor gate 115 that transfers charges of the photoelectric conversion unit, and the amplifier transistor gate 116, the selection transistor gate 117, and the reset transistor gate 118 (which are not shown), which are connected by multilayer wiring in the wiring layer 113.

When the reflective layer 114 is provided below the photoelectric conversion unit 112, light emitted from the biological substance S can be reflected and returned to the photoelectric conversion unit 112, and the photoelectric conversion unit 112 can utilize a large amount of light. As a result, it is possible to improve detection accuracy.

Here, in the second embodiment of FIG. 10, arrangement of the reflective layer 114 is not particularly limited as long as the reflective layer 114 is arranged between the photoelectric conversion unit 112 and the wiring layer 113 and is provided below the photoelectric conversion unit 112. Although not shown, the reflective layer 114 can also be arranged below the wiring layer 113.

### (3) Third Embodiment

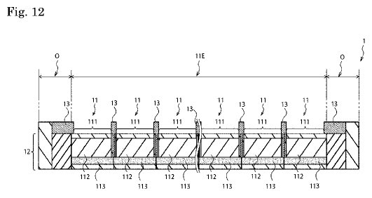

FIG. 12 is a schematic end view schematically showing a third embodiment of the biological substance detection chip 1 according to the present technology. The third embodiment includes a partition wall 13 between pixels in the biological substance detection chip 1 according to the first embodiment. When the partition wall 13 is provided, it is possible to prevent light from leaking between pixels, and it is possible to further improve detection accuracy.

The material constituting the partition wall 13 is not particularly limited as long as the effects of the present technology are not impaired. For example, the partition wall 13 can be made of a metal or the like, and for example, tungsten (W), aluminum (Al), copper (Cu), titanium (Ti) or the like can be used as the metal.

Here, although not shown, also in the biological substance detection chip 1 according to the third embodiment, as in the second embodiment shown in FIG. 10, of course, it is possible to provide the reflective layer 114. The same applies to the following embodiments.

### (4) Fourth Embodiment

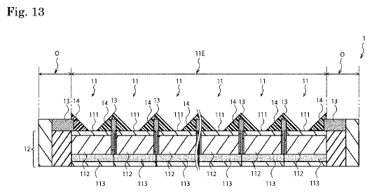

FIG. 13 is a schematic end view schematically showing a fourth embodiment of the biological substance detection chip 1 according to the present technology. The fourth embodiment includes a light guiding unit 14 that guides light emitted from the holding surface 111 in a direction other than a direction of the photoelectric conversion unit 112 in the direction of the photoelectric conversion unit 112. In the fourth embodiment, the chip has a structure in which a refractive member is used for the light guiding unit 14, and light emitted from the biological substance S can be guided in a direction of the photoelectric conversion unit 112.

The material used for the refractive member can be freely selected and used as long as the effects of the present technology are not impaired. For example, silicon oxide (SiO₂), silicon nitride (Si₃N₄), silicon oxynitride (SiON), a high-refractive-index resin and the like can be used.

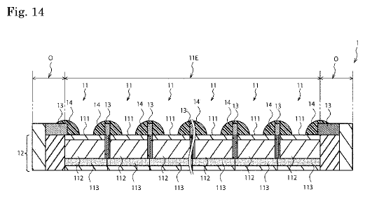

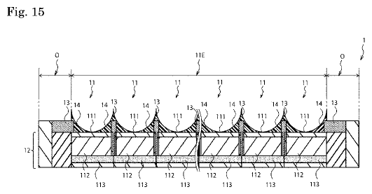

The specific form of the refractive member is not limited to the triangular structure in the fourth embodiment shown in FIG. 13, and can be freely designed according to the refractive index of the material used, the size of the pixel, the size of the biological substance S and the like. For example, it can be designed in a form such as a first modified example of the fourth embodiment shown in FIG. 14 and a second modified example of the fourth embodiment shown in FIG. 15.

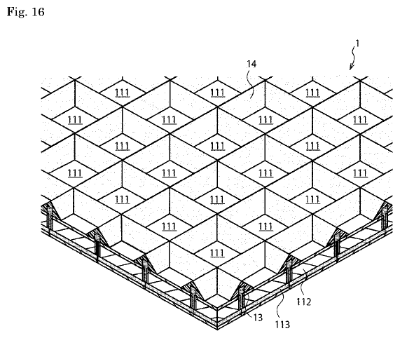

The planar layout of the light guiding unit 14 using the refractive member and the reflective member to be described below is not particularly limited, and for example, the layout can be as shown in the schematic perspective view schematically showing the planar layout of the fourth embodiment of the biological substance detection chip 1 according to the present technology of FIG. 16. In the example of FIG. 16, the opening is rectangular, but the shape is not limited thereto, and although not shown, for example, it can be designed in a circular shape, an oval shape or the like.

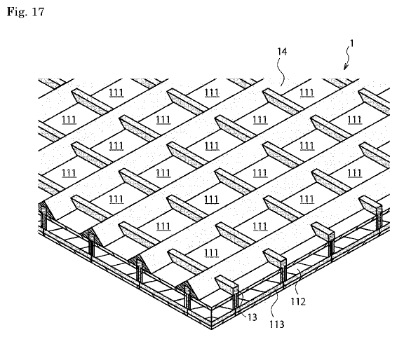

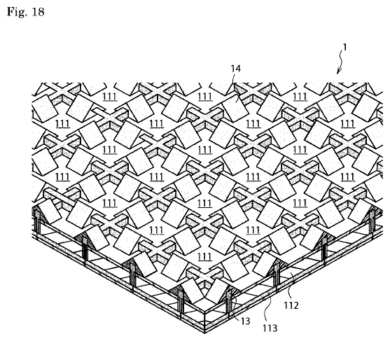

In the detection of the biological substance S, since a sample liquid containing the biological substance S or a reagent may flow, or a cleaning liquid may flow, unevenness may occur due to a step of the refractive member or the reflective member to be described below. Therefore, for example, as shown in the schematic perspective view schematically showing a first modified example of the planar layout of the fourth embodiment of the biological substance detection chip 1 according to the present technology of FIG. 17, the light guiding unit 14 can be formed only on one side in the vertical and horizontal directions. In addition, for example, as shown in the schematic perspective view schematically showing a first modified example of the planar layout of the fourth embodiment of the biological substance detection chip 1 according to the present technology of FIG. 18, it is possible to form a layout in which the light guiding unit 14 is removed from the part that the partition wall 13 intersects. By devising the layout of the light guiding unit 14 in this manner, unevenness in the sample liquid, the reagent, the cleaning liquid and the like can be minimized, and as a result, it is possible to further improve detection accuracy.

### (5) Fifth Embodiment

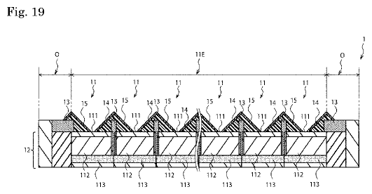

FIG. 19 is a schematic end view schematically showing a fifth embodiment of the biological substance detection chip 1 according to the present technology. The fifth embodiment is an example in which an anti-reflection structure 15 is provided on the surface of the light guiding unit 14 composed of a refractive member in the biological substance detection chip 1 according to the fourth embodiment shown in FIG. 13. When the anti-reflection structure 15 is provided on the surface of the light guiding unit 14, it is possible to prevent light from the biological substance S from being reflected on the surface of the light guiding unit 14. As a result, the light guiding unit 14 increases the amount of light guided to the photoelectric conversion unit 112, and it is possible to further improve detection accuracy.

The specific structure of the anti-reflection structure 15 is not particularly limited as long as it is a structure that can prevent reflection of light. For example, a thin film structure, a moth-eye structure and the like using a refractive material different from that of the light guiding unit 14 can be used.

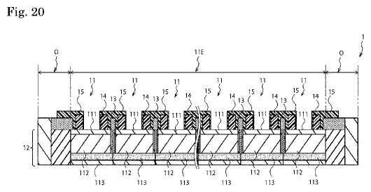

The specific form of the light guiding unit 14 composed of a refractive member and the anti-reflection structure 15 is not limited to the structure as in the fifth embodiment shown in FIG. 19, and can be freely designed according to the refractive index of the material used, the size of the pixel, the size of the biological substance S, and the like. For example, it can be designed in a form such as a first modified example of the fifth embodiment shown in FIG. 20.

Here, while an example in which the partition wall 13 is provided is shown in the fourth embodiment and the fifth embodiment shown in FIGS. 13 to 20, the partition wall 13 is not essential, and the light guiding unit 14 can be provided between the pixels 11 without providing the partition wall 13.

### (6) Sixth Embodiment

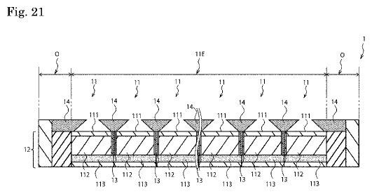

FIG. 21 is a schematic end view schematically showing a sixth embodiment of the biological substance detection chip 1 according to the present technology. In the sixth embodiment, the chip has a structure in which a reflective member is used for the light guiding unit 14 and light emitted from the biological substance S can be guided in a direction of the photoelectric conversion unit 112.

The material used for the reflective member can be freely selected and used as long as the effects of the present technology are not impaired. For example, aluminum (Al), tungsten (W) and the like can be used.

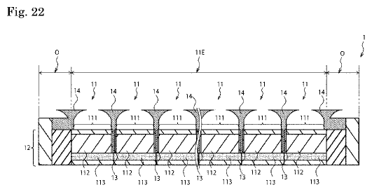

The specific form of the reflective member is not limited to the form as in the sixth embodiment shown in FIG. 21 as long as it is a form in which light from the biological substance S held on the holding surface 111 can be guided to the photoelectric conversion unit 112, and can be freely designed according to the size of the pixel, the size of the biological substance S and the like. For example, it can be designed in a form such as a first modified example of the sixth embodiment shown in FIG. 22.

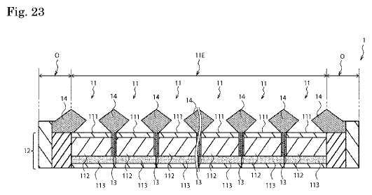

In addition, the upper surface of the light guiding unit 14 composed of a reflective member does not have to be flat, and for example, as in the second modified example of the sixth embodiment shown in FIG. 23, it can be designed to be inclined toward the holding surface 111. By designing in this manner, the sample liquid containing the biological substance S, the reagent and the like can be guided to the holding surface 111, and efficient supply of the sample liquid can be promoted. In addition, it is possible to prevent the sample liquid from remaining on the light guiding unit 14.

Here, while an example in which the partition wall 13 is provided is shown in the sixth embodiment shown in FIGS. 21 to 23, the partition wall 13 is not essential, and the light guiding unit 14 can be provided between the pixels 11 without providing the partition wall 13. In addition, when the partition wall 13 is provided, the light guiding unit 14 composed of a reflective member can be integrated with the partition wall 13, for example, as in the first modified example and the second modified example of the sixth embodiment of FIGS. 22 and 23.

### (7) Seventh Embodiment

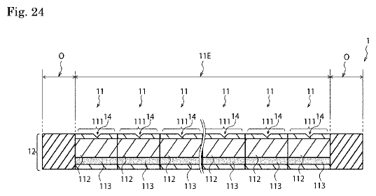

FIG. 24 is a schematic end view schematically showing a seventh embodiment of the biological substance detection chip 1 according to the present technology. In the seventh embodiment, the chip has a structure in which, as the light guiding unit 14, a recess is formed in the holding surface 111, the biological substance S is held in the recess, and thus light emitted from the biological substance S can be guided in a direction of the photoelectric conversion unit 112.

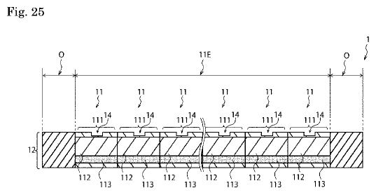

The form of the recess is not particularly limited as long as the effects of the present technology are not impaired, and can be freely designed according to the size of the pixel, the size of the biological substance S and the like. For example, it can be designed in a form such as a first modified example of the seventh embodiment shown in FIG. 25.

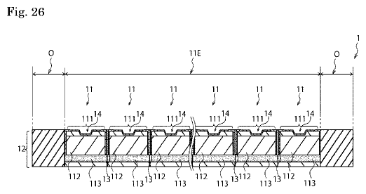

In addition, as in the second modified example of the seventh embodiment shown in FIG. 26, when the partition wall 13 is provided between the pixels 11, it is possible to prevent light from leaking between pixels, and it is possible to further improve detection accuracy.

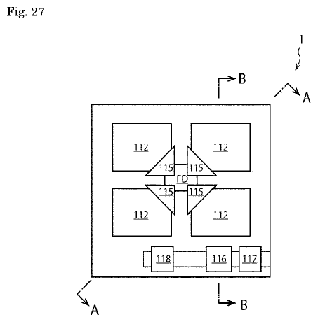

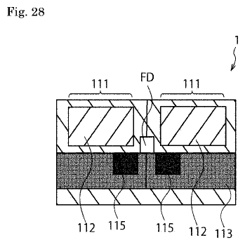

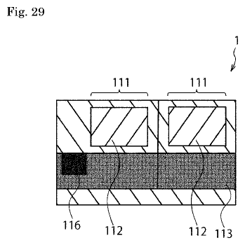

FIG. 27 is a schematic bottom view schematically showing the biological substance detection chip 1, which is an example of the embodiments so far, when viewed from the side of the wiring layer 113. In FIG. 27, the wiring and the substrate are omitted to show the photoelectric conversion unit 112, the transistor and the like. FIG. 28 is a schematic end view taken along the line A-A, schematically showing the biological substance detection chip 1, which is an example of the embodiments so far, and FIG. 29 is a schematic end view taken along the line B-B, schematically showing the biological substance detection chip 1, which is an example of the embodiments so far.

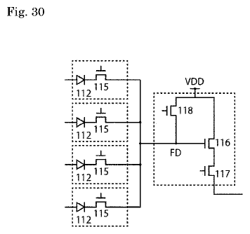

FIG. 30 is an equivalent circuit diagram showing an example of a configuration of FIG. 27. The pixel signal of the photoelectric conversion unit 112 is output in a time division manner by the pixel circuit of a shared amplifier gate 116, selection gate 117, and reset gate 118 when the transfer transistor gate 115 is driven in a time division manner. These are structures in which four pixels share one pixel circuit. In addition, although not shown, the embodiments so far may have a configuration in which the pixel circuit is not shared.

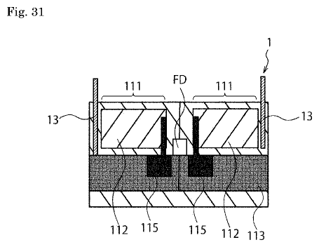

FIG. 31 is a modified example of FIG. 28. As shown in FIG. 31, the partition wall 13 can be provided between the pixels 11. When the partition wall 13 is provided, it is possible to prevent light from leaking between the pixels 11, and it is possible to further improve detection accuracy. Here, the partition wall 13 may partially penetrate to the wiring layer 113. In addition, the partition wall 13 can be provided for each pixel 11, or can be provided for each unit of accumulated pixels 11 (for example, in units of four pixels) when the plurality of pixels 11 are accumulated.

In addition, in order to efficiently extract charges from the photoelectric conversion unit 112, as shown in FIG. 31, the transfer transistor gate 115 can be embedded in the photoelectric conversion unit 112. Since the transfer transistor gate 115 has a light-shielding property, it is possible to prevent light from leaking between the pixels 11 as in the partition wall 13, and it is possible to further improve detection accuracy.

### (8) Eighth Embodiment

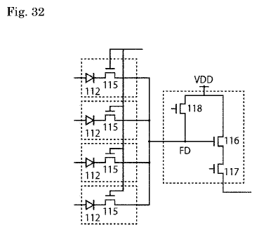

The biological substance detection chip 1 according to an eighth embodiment is an example in which pixel signals of a plurality of photoelectric conversion units 112 are added and output. For example, as shown in FIG. 32, signals from a plurality of pixels can be added by performing switching so that the transfer transistor gates 115 are driven at the same time. By this switching, the spatial resolution of detection is lowered, but the sensitivity can be improved and the temporal resolution can be increased.

Here, in the eighth embodiment, by devising the structure of the biological substance detection chip 1, signal charges from the plurality of pixels 11 can be added and output, but for example, according to calculation, of course, it is possible to add and output signal charges from the plurality of pixels 11.

The eighth embodiment is an example in which 4 pixels are accumulated, but the number of accumulated pixels is not particularly limited. In addition, although not shown, it is possible to change the number of pixels accumulated for each area on one chip. More specifically, it is possible to divide the accumulation area on one chip, for example, an area in which 4 pixels are accumulated, an area in which 8 pixels are accumulated, and an area in which 16 pixels are accumulated.

<3. Biological Substance Detection Device 2>

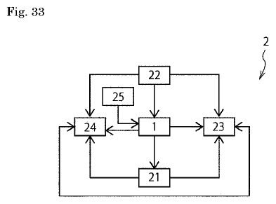

FIG. 33 is a block diagram showing a concept of the biological substance detection device 2 according to the present technology. The biological substance detection device 2 according to the present technology includes at least the above biological substance detection chip 1 according to the present technology and an analysis unit 21. In addition, according to their purpose, a light emission unit 22, a storage unit 23, a display unit 24, a temperature control unit 25 and the like can be provided. Hereinafter, respective units will be described. Here, since the biological substance detection chip 1 is as described above, descriptions thereof will be omitted here.

(1) Analysis Unit 21

In the analysis unit 21, optical information acquired by the biological substance detection chip 1 is analyzed. For example, based on the optical information acquired by the biological substance detection chip 1, checking whether the biological substance S is present, checking whether there is an interaction with the biological substance S, and screening of medicinal components and the like are performed.

Here, the analysis unit 21 may be implemented in a personal computer or a CPU, or may be stored as a program in a hardware resource including a recording medium (for example, a nonvolatile memory (a USB memory), an HDD, or a CD) and the like, and can function by a personal computer or a CPU.

(2) Light Emission Unit 22

The biological substance detection device 2 according to the present technology can include, for example, the light emission unit 22 for emitting excitation light. The light emission unit 22 emits light to the biological substance S held on the holding surface 111 of the biological substance detection chip 1. Here, in the biological substance detection device 2 according to the present technology, the light emission unit 22 is not essential, and light can be emitted to the biological substance S using an external light emission device or the like.

The type of light emitted from the light emission unit 22 is not particularly limited, but in order to reliably generate fluorescence or scattered light from microparticles, light having a constant light direction, wavelength, and light intensity is desirable. As an example, a laser, an LED and the like may be exemplified. When a laser is used, the type thereof is not particularly limited, and an argon ion (Ar) laser, a helium-neon (He-Ne) laser, a dye laser, a krypton (Cr) laser, a semiconductor laser, and a solid laser in which a semiconductor laser and a wavelength conversion optical element are combined can be used alone or two or more thereof can be freely used in combination.

According to their purpose, a plurality of light emission units 22 may be provided. For example, one light emission unit 22 may be provided for each pixel 11 of the biological substance detection chip 1. In addition, when a substrate in which light emitting elements such as LEDs are arranged at positions corresponding to the pixels 11 of the biological substance detection chip 1 is laminated on the biological substance detection chip 1, light can be emitted to the biological substance S.

(3) Storage Unit 23

The biological substance detection device 2 according to the present technology can include the storage unit 23 in which various types of information are stored. The storage unit 23 can store all items related to detection such as optical data acquired by the biological substance detection chip 1, analysis data generated by the analysis unit 21, and optical data emitted by the light emission unit 22.

In the biological substance detection device 2 according to the present technology, the storage unit 23 is not essential, and an external storage device may be connected. As the storage unit 23, for example, a hard disk or the like can be used.

(4) Display Unit 24

The biological substance detection device 2 according to the present technology can include the display unit 24 that displays various types of information. The display unit 24 can display all items related to detection such as optical data acquired by the biological substance detection chip 1, analysis data generated by the analysis unit 21, optical data emitted by the light emission unit 22, data stored in the storage unit 23 and the like.

In the biological substance detection device 2 according to the present technology, the display unit 24 is not essential, and an external display device may be connected. As the display unit 24, for example, a display, a printer or the like can be used.

(5) Temperature Control Unit 25

The biological substance detection device 2 according to the present technology can include the temperature control unit 25 that keeps the biological substance S held on the holding surface 111 of the biological substance detection chip 1 at a predetermined temperature and heats or cools it to a predetermined temperature. For example, when the biological substance S is an enzyme, the temperature control unit 25 can control the temperature so that an optimal temperature is maintained. In addition, when the biological substance S is a nucleic acid, and the presence of hybridization is detected using the present technology, the temperature control unit 25 can perform control so that the temperature range in which hybridization is possible is maintained. As the temperature control unit 25, a thermoelectric element such as a Peltier element can be used.

According to their purpose, a plurality of temperature control units 25 may be provided. For example, one temperature control unit 25 may be provided for each pixel 11 of the biological substance detection chip 1. In addition, when a substrate in which thermoelectric elements are arranged at positions corresponding to the pixels 11 of the biological substance detection chip 1 is laminated on the biological substance detection chip 1, the temperature of the biological substance S can be controlled.

Here, in the biological substance detection device 2 according to the present technology, the temperature control unit 25 is not essential, and the temperature of the biological substance S can be controlled using an external temperature control device or the like.

<4. Biological Substance Detection System 3>

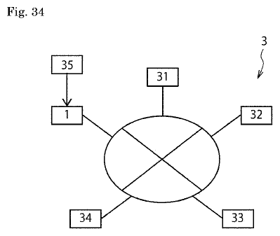

FIG. 34 is a block diagram showing a concept of the biological substance detection system 3 according to the present technology. The biological substance detection system 3 according to the present technology includes at least the above biological substance detection chip 1 according to the present technology and an analysis device 31. In addition, according to their purpose, a light emission device 32, a storage device 33, a display device 34, a temperature control device 35 and the like can be provided.

The biological substance detection chip 1 and respective devices can be connected via a wired or wireless network. Here, since details of respective devices are the same as details of respective units of the biological substance detection device 2 of the present technology described above, descriptions thereof will be omitted here.

Here, in the present technology, the following configurations can be used.

(1) A biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit.

(2) A biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit.

(3) The biological substance detection chip according to (2),

wherein a wiring layer is provided below the photoelectric conversion unit.

(4) The biological substance detection chip according to any one of (1) to (3),

wherein a reflective layer is provided below the photoelectric conversion unit.

(5) The biological substance detection chip according to any one of (2) to (4),

wherein the light guiding unit is composed of a refractive member and/or a reflective member provided between the pixels.

(6) The biological substance detection chip according to any one of (2) to (5),

wherein the light guiding unit is a recess formed on the holding surface.

(7) The biological substance detection chip according to any one of (1) to (6),

wherein signal charges from the plurality of pixels are added and output.

(8) The biological substance detection chip according to any one of (1) to (7),

wherein the biological substance is one or more biological substances selected from among nucleic acids, proteins, cells, microorganisms, chromosomes, ribosomes, mitochondria, organelles (cell organelles), and complexes thereof.

(9) A biological substance detection device, including:

a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below

the photoelectric conversion unit; and

an analysis unit that analyzes electrical information acquired by the biological substance detection chip.

(10) A biological substance detection device, including:

a biological substance detection chip which is composed of a plurality of pixels, in which the pixel includes a holding surface on which a biological substance is held and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit; and

an analysis unit that analyzes electrical information acquired by the biological substance detection chip.

(11) A biological substance detection system, including:

a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit; and

an analysis device that analyzes electrical information acquired by the biological substance detection chip.

(12) A biological substance detection system, including:

a biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit, and

an analysis device that analyzes electrical information acquired by the biological substance detection chip.

### REFERENCE SIGNS LIST

1 Biological substance detection chip

11 Pixel

S Biological substance

111 Holding surface

12 Semiconductor substrate

112 Photoelectric conversion unit

113 Wiring layer

114 Reflective layer

13 Partition wall

14 Light guiding unit

15 Anti-reflection structure

115 Transfer transistor gate

116 Amplifier transistor gate

117 Selection transistor gate

118 Reset transistor gate

21 Analysis unit

22 Light emission unit

23 Storage unit

24 Display unit

25 Temperature control unit

31 Analysis device

32 Light emission device

33 Storage device

34 Display device

35 Temperature control device

1. A biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit.

2. A biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit.

3. The biological substance detection chip according to claim 2,
wherein a wiring layer is provided below the photoelectric conversion unit.

4. The biological substance detection chip according to claim 1,
wherein a reflective layer is provided below the photoelectric conversion unit.

5. The biological substance detection chip according to claim 2,
wherein the light guiding unit is composed of a refractive member and/or a reflective member provided between the pixels.

6. The biological substance detection chip according to claim 2,
wherein the light guiding unit is a recess formed on the holding surface.

7. The biological substance detection chip according to claim 1,
wherein signal charges from the plurality of pixels are added and output.

8. The biological substance detection chip according to claim 1,
wherein the biological substance is one or more biological substances selected from among nucleic acids, proteins, cells, microorganisms, chromosomes, ribosomes, mitochondria, organelles (cell organelles), and complexes thereof.

9. A biological substance detection device, comprising:
a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit; and
an analysis unit that analyzes electrical information acquired by the biological substance detection chip.

10. A biological substance detection device, comprising:
a biological substance detection chip which is composed of a plurality of pixels, in which the pixel includes a holding surface on which a biological substance is held and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit; and
an analysis unit that analyzes electrical information acquired by the biological substance detection chip.

11. A biological substance detection system, comprising:
a biological substance detection chip which is composed of a plurality of pixels, in which the pixel includes a holding surface on which a biological substance is held, a photoelectric conversion unit that is provided below the holding surface and provided on a semiconductor substrate, and a wiring layer that is provided below the photoelectric conversion unit; and
an analysis device that analyzes electrical information acquired by the biological substance detection chip.

12. A biological substance detection system, comprising:
a biological substance detection chip which is composed of a plurality of pixels, in which the pixel includes a holding surface on which a biological substance is held, and a photoelectric conversion unit that is provided below the holding surface and provided on a semiconductor substrate, and which includes a light guiding unit that guides light emitted in a direction other than a direction of the photoelectric conversion unit from the holding surface in the direction of the photoelectric conversion unit, and
an analysis device that analyzes electrical information acquired by the biological substance detection chip.
